(19) United States
(12) Patent Application Publication
(10) Pub. No.: US 2017/0182108 A1
(43) Pub. Date: Jun. 29, 2017

(54) FERMENTED FOOD EXTRACT COMPOSITION

(71) Applicant: Shinshu University, Matsumoto-shi, Nagano (JP)

(72) Inventor: Kozo Nakamura, Nagano (JP)

(73) Assignee: Shinshu University, Matsumoto-shi, Nagano (JP)

(21) Appl. No.: 15/129,473

(22) PCT Filed: Mar. 27, 2015
(86) PCT No.: PCT/JP2015/059578

§ 371 Date: Sep. 27, 2016

(30) Foreign Application Priority Data
Mar. 28, 2014 (JP) 2014-070226

(51) Int. Cl. A61K 36/70; A23L 33/105; A61K 31/685; A61K 31/19; A61K 31/221; A61K 31/197

(52) U.S. Cl. CPC A61K 36/70; A61K 31/221; A61K 31/197; A61K 31/685; A61K 31/19; A23L 33/105; A23V 2002/00

(57) ABSTRACT

Provided is a fermented food extract that has a blood pressure-lowering action and is useful as a functional health food or pharmaceutical composition. This fermented food extract is obtained by: subjecting a fermented food, preferably a fermented fagopyrum esculentum lyophilisate, to acetone extraction; eluting the fermented fagopyrum esculentum acetone extract using a solid phase extract cartridge filled with a resin to which a phenyl group is bonded, with formic acid-containing water used as an eluent; and fractionating and purifying the eluate using a column (PFP column) filled with a carrier to which a pentafluorophenyl group is bonded, with formic acid/methanol-containing water used as a mobile phase. This functional health food or pharmaceutical composition contains said extract or at least a part thereof as an active ingredient.

### TECHNICAL FIELD

The present invention relates to an extract composition extracted from fermented foods, etc., for example, fermented buckwheat, etc. More specifically, the present invention relates to a fermented food extract composition comprising at least acetylcholine and propionylcholine, which shows blood pressure-lowering effect and vasodilatory effect upon oral administration, as well as to an extraction method thereof.

In addition, the present invention relates to a food or pharmaceutical composition comprising said extract composition as an active ingredient.

### BACKGROUND ART

The present inventors have promoted research on fermented foods, in particular a fermentation product of juice of germinated buckwheat, and found that lyophilized powder of said fermentation product exhibits an ACE inhibitory effect; and they have reported functional foods and functional beverages consisting of said powder (Patent Document 1), and further reported a fractionation method of this ACE inhibitor and fractions separated by said method (Patent Document 2).

Then, the present inventors have developed a lactic acid fermentation product of buckwheat plant-pulverized products having higher functionality (fermented buckwheat), and they have further prompted the research with the aim of isolating and purifying active ingredients having blood pressure-lowering effect and vasodilatory effect contained in said fermentation products, and succeeded in finding a fermented buckwheat extract containing several novel peptides, and filed a patent application thereof (Patent Document 3).

Meanwhile, to date, there have been several reports regarding buckwheat-derived compounds having a vasodilatory effect, and compositions containing them (Patent Documents 4-7).

The present inventors have further conducted intensive research on active ingredients having blood pressure-lowering effect and vasodilatory effect contained in the fermented buckwheat, and fractionated a component with vasodilatory effect which is different from the active ingredients reported in the above Patent Document 3.

Then, as a result of identity confirmation of the component contained in said fraction, they have found that said component comprises a quaternary alkylammonium compound mainly composed of a plurality of choline esters including at least acetylcholine and propionylcholine.

Among choline esters, acetylcholine is known to be an essential substance for life activity as a neurotransmitter in mammal. In addition, in 1929, a hypotensive substance different from histamine was isolated from the spleen of horse, and this active substance was chemically identified as acetylcholine (Non-patent Document 1).

Thus, the presence of acetylcholine in animal tissues has been known for a long time. Furthermore, a hypotensive substance in ergot has been revealed to be acetylcholine, and fungi have been confirmed to produce acetylcholine (Non-patent Document 2)

Acetylcholine is contained in edible plants, edible fungi, royal jelly and milk, etc., and acetylcholine is also present in Bacillus subtilis and yeasts; there is also a report that production of acetylcholine by Lactobacillus plantarum, i.e., a kind of lactic acid bacteria, has been confirmed by biological assays (Non-patent Documents 3-7).

Other than acetylcholine, several choline esters have also been discovered. Propionylcholine that has a propionyl group with a carbon chain longer than that of acetyl group by one carbon has been found from bovine spleen in 1953 (Non-patent Documents 8 and 9).

Then, production of propionylcholine has been confirmed in the semen of ox, in the hermal lymph nodes and smooth muscles of European crayfish, American horseshoe crabs, common Europe cockles, soft-shell clams, mussels, Swan mussels (Anodonta cygnea), and escargot de Bourgogne (Helix pomatia), in the electricity generation tissue culture of Torpedo, as well as in croton, mung beans, Chinese plantain, poplar, and birch (Non-patent Documents 10-13).

However, to date, presence of propionylchone in fermented foods has not yet been reported or even suggested.

As for butyrylcholine, it was discovered from a brain extract in 1954 (Non-patent Document 14), and has been shown to be present in arthropod and mollusk together with acetylcholine and propionylcholine (Non-patent Document 11).

Furthermore, in addition to propionylcholine and butyrylcholine, several choline esters have been identified from mollusk. For example, structure determination has been conducted on urocanoyl choline from a kind of Troschel's Murex, ββ-dimethyl acroyl choline (senecioyl choline) from a kind of Thais (Reishia) bronni, acroyl choline from a kind of Buccinidae, and imidazole propionylcholine from a kind of Thais (Reishia) bronni (Non-patent Documents 15-18).

However, to date, presence of a choline ester in fermented foods has not been chemically confirmed, and its extraction and fractionation methods have not yet been reported or even suggested.

### PRIOR ART DOCUMENTS

### Patent Documents

[Patent Document 1] JP A 2005-304355
[Patent Document 2] JP A 2008-239498
[Patent Document 3] JP A 2012-162503
[Patent Document 4] JP A 2007-254410
[Patent Document 5] JP A 2006-76903
[Patent Document 6] JP A 2006-76904
[Patent Document 7] JP A 5-97798

### [Non-Patent Documents]

[Non-patent Document 1] Dale H H, Dudley H W, The presence of histamine and acetylcholine in the spleen of the ox and the horse. J Physiol 68:97-123, 1929.
[Non-patent Document 2] Ewins A J. Acetylcholine, a new active principle of ergot. Biochem J 8:44-49, 1914.
[Non-patent Document 3] Yoshie Momonoki: Acetylcholine in plants, Chemical Regulation in Plants 30 (1) 49-61 (1995) (In Japanese).
[Non-patent Document 4] Koichiro Kawashima: Roots of acetylcholine and non-neuronal acetylcholine, Basic Aging Research 34 (4), 12-24 (2010) (In Japanese.).
[Non-patent Document 5] Masato Shinoda et al.: Blood flow-increasing factor in royal jelly, Pharmaceutical Journal 98 (2), 139-145 (1978) (In Japanese).
[Non-patent Document 6] Whittaker V P. Acetylcholine in Milk. Nature 181:856-857, 1958.
[Non-patent Document 7] Stephenson M, Rowatt E. The production of acetylcholine by a strain of Lactobacillus plantarum. J Gen Microbiol 1(3):279-298, 1947.
[Non-patent Document 8] Banister J, Whittaker V P, Wijesundera S. The occurrence of homologues of acetylcholine in ox spleen. J Physiol 121 (1) :55-71, 1953.
[Non-patent Document 9] Gardiner J E, Whittaker V P, The identification of propionylcholine as a constituent of ox spleen. Biochem J 58 (1) :24-29, 1954.
[Non-patent Document 10] Bishop M R, Sastry B V, Stavinoha W B Identification of acetylcholine and propionylcholine in bull spermatozoa by integrated pyrolysis, gas chromatography and mass spectrometry. Biochem Biophys Acta 500 (2):440-444, 1977.
[Non-patent Document 11] Wolfgang W, Jutta N, Dettmar W. Distribution of cholinesters and cholinesterases in haemolymphs and smooth muscles of molluscs. Comp Biochem Phys C 61 (1) :121-131, 1978.
[Non-patent Document 12] O'Regan S. The synthesis, storage, and release of propionylcholine by the electric organ of Torpedo marmorata. J Neurochem 39 (3) :764-772, 1982.
[Non-patent Document: 13] Miural G A, Shin T M. Identification of proprionylcholine in higher plants. Physiol Plant 62:341-343, 1984.
[Non-patent Document 14] Holtz P, Schumann H J. Butyrylcholine in brain extracts. Naturwissenschaften 41:306, 1954.
[Non-patent Document 15] Erspamer V, Benati O. Identification of murexine as β-[imidazolyl-(4)]acrylcholine. Science 117:161-162, 1953.
[Non-patent Document 16] Keyl M J, Michaelson I A, Whittaker V P. Physiologically active choline esters in certain marine gastropods and other invertebrates. J Physiol 139:434, 1957.
[Non-patent Document 17] Whittaker V P. Acrylylcholine: a new naturally occurring pharmacologically active choline ester from Buccinum undatum. Biochem Pharmacol 1(4) :342-346, 1959.
[Non-patent Document 18] Roseghini M. Occurrence of dihydromurexine (imidazole propionylcholine) in the hypobranchial gland of Thais (purpura) haemastoma. Experientia 27(9):1008-1009, 1971.

### SUMMARY OF THE INVENTION

### Problems to Be Solved by the Invention

The object of the present invention is to provide a novel fermented food extract having blood pressure-lowering effect and vasodilatory effect which is useful as functional health foods or pharmaceutical compositions, and a functional health food or a pharmacetical composition comprising said extract or at least a part of an active ingredient contained in said extract as an active ingredient.

### Means for Solving the Problems

The present inventors have conducted extensive research to solve the above problems, and accomplished the present invention. Namely, the present inventors have succeeded in separating active ingredients that exert very significant blood pressure-lowering effect and vasodilatory effect, as a result of extensive research on components contained in fermented foods, in particular, in a lactic acid-fermentation product of buckwheat plant (fermented buckwheat), and also succeeded in identifying some of the active ingredients, and thus completed the present invention.

Specifically, the present invention relates to:
[1] a fermented food extract composition comprising at least acetylcholine and propionylcholine, which shows blood pressure-lowering effect and vasodilatory effect upon oral administration,
[2] the extract composition according to [1], wherein the fermented food is one type selected from the group of fermented buckwheat, black vinegar, yogurt, natto, and Japanese sake,
[3] the extract composition according to [2], wherein the fermented food is fermented buckwheat, and
[4] the extract composition according to [1], further comprising at least one type selected from the group of butyrylcholine, lactylcholine, levulinylcholine, carnitine, methyl carnitine, trimethylglycine (betaine), methyl trimethylglycine, ethyl trimethylglycine, propionyl trimethylglycine and phosphocholine.

In addition, the present invention relates to:

[5] a food or pharmaceutical composition comprising, as an active ingredient, the extract composition according to any one of [1] to [4],
[6] the food or pharmaceutical composition according to [5], further comprising, as an active ingredient, at least one type selected from the group of tyrosine and y-aminobutyric acid (GABA) and
[7] the food or pharmaceutical composition according to [5] or [6] , further comprising at least one type selected from the group of lactic acid, acetic acid, and citric acid.

Furthermore, the present invention relates to:
[8] a method of producing the fermented food extract composition according to [1], characterized in that the method consists of the steps comprising:
(1) a step wherein a lyophilizate or concentrate of fermented buckwheat is suspended in acetone, stirred by shaking, then centrifuged to obtain a supernatant, which is then vacuum concentrated (step 1),
(2) a step wherein said vacuum concentrate is eluted using a solid phase extraction cartridge filled with a carrier to which a phenyl group is bonded, with acid-containing water as an eluent (step 2),
(3) a step wherein said eluate is fractionated and purified using a column (PFP column) filled with carrier to which a pentafluorophenyl group is bonded, with acidic methanol-containing water as a mobile phase (step 3), and
[9] the production method according to [8], characterized in that:
(1) in step 2, a solid phase extraction cartridge (GL Science InertSep® PH) is used as the solid phase extraction cartridge, and 0.01% formic acid-containing water is used as the eluent,
(2) in step 3, a PET column (YMC-Triart PFP) is used as the reverse phase column, and water containing 0.01% formic acid-33% methanol is used as the mobile phase.

Furthermore, the present invention relates to:
[10] a fermented food extract composition produced by the production method according to [8] or [9], characterized in that it comprises at least acetylcholine and propionylcholine, and shows blood pressure-lowering effect and vasodilatory effect upon oral administration.

### Advantageous Effects of the Invention

The novel fermented food extract of the present invention has significant blood pressure-lowering effect and vasodilatory effect, which is useful as an active ingredient of functional health foods or pharmaceutical compositions.

### BRIEF DESCRIPTION OF DRAWINGS

FIG. 1 is a chart of detection peaks showing changes in the elution behavior of acetylcholine with changes in the methanol content, in the examination test of component separation conditions for solid phase extract of fermented buckwheat using PFP column.

FIG. 2 is a chart of detection peaks showing changes in the elution behavior of acetylcholine with changes in the acid content, in the examination test of component separation conditions for solid phase extract of fermented buckwheat using PFP column.

FIG. 3 is similarly a chart of detection peaks showing changes in the elution behavior of acetylcholine in the purified products of fermented buckwheat with changes in the methanol content under the fixed condition of acid content (0.01%), in the examination test of component separation conditions for solid phase extract of fermented buckwheat.

FIG. 4 is similarly a chart of detection peaks showing changes in the elution behavior of acetylcholine in the purified products of fermented buckwheat with changes in the methanol content under the fixed condition of acid content (0.01%), when the methanol content is finely varied (38%, 36%, 33%) in the examination test of component separation conditions for solid phase extract of fermented buckwheat.

FIG. 5 is similarly a chart of detection peaks showing changes in the elution behavior of acetylcholine in the purified products of fermented buckwheat with changes in the separation temperature, when the separation is examined by setting the separation temperature in water containing 0.01% formic acid-33% methanol at 30° C. and 40° C. in the examination test of component separation conditions for solid phase extract of fermented buckwheat.

FIG. 6 is a graph showing the results of measurement test of isometric tension in blood vessels using identified choline compounds.

FIG. 7 is a graph showing changes in the systolic blood pressure after administration of identified choline compounds.

FIG. 8-1 is a mass spectrum of lactylcholine in LC-MS analysis of gel filtration products.

FIG. 8-2 is a mass spectrum of lactylcholine in LC-MS/MS analysis of gel filtration products

FIG. 9-1 is similarly a mass spectrum of levulinylcholine in LC-MS analysis of gel filtration products.

FIG. 9-2 is similarly a mass spectrum of levulinylcholine LC-MS/MS analysis of gel filtration products.

### MODES FOR CARRYING OUT THE INVENTION

“Fermented buckwheat”, which is a buckwheat plant that has been subjected to lactic acid fermentation, has an excellent blood pressure-lowering effect for a spontaneously hypertensive rat (SHR).

The present inventors have conducted research on hypotensive substances in this fermented buckwheat, and found a fermented buckwheat extract containing several novel peptides, and filed a patent application thereof (Patent Document 3).

The inventors have further promoted research on vasodilating components in the fermented buckwheat, developed a novel method of fractionation and purification, and succeeded in fractionating and purifying the vasodilating components in said fermented buckwheat.

The fermented buckwheat used in the fractionation and purification of the present invention is acidic with pH 3.6, and it shows a vasodilatory effect from 0.5 μg/mL for the blood vessel specimens of thoracic aorta of 12-week-old male SHRs, with EC₅₀ of 8.30 μg/mL.

When a sodium hydroxide solution is added to this fermented buckwheat and allowed to stand at room temperature and pH 12, the vasodilatory activity was lost within 0.5 h.

In addition, when the vasodilatory effect of organic solvent extracts of a fermented buckwheat lyophilizate was examined, an ethyl acetate extract did not have the vasodilatory effect, and ethanol and acetone extracts had the vasodilatory effect, and acetone extract had the strongest vasodilatory effect.

From the above results, it was found that vasodilating components of fermented buckwheat should be purified and administered under acidic or neutral conditions, and that they can be effectively extracted with acetone.

Next, purification conditions of vasodilating components by chromatography were investigated. First, we compared the activity of acetone crude extracts of a fermented buckwheat lyophilizate purified by gel filtration chromatography. As a result, vasodilatory effect was observed in fractions containing a substance with relatively low molecular weight determined from the elution time.

Since vasodilating components of fermented buckwheat were inactivated under basic conditions, we investigated the purification by cation exchange resin operated under acidic conditions. As a result, a relatively strong vasodilatory effect was observed in a hydrochloric acid elution fraction in the strong acid cation exchange resin treatment. This result was presumably due to the interaction of the vasodilating components of fermented buckwheat having a positive charge with sulfonate groups that are an exchange group having a negative charge on the resin.

However, adsorption of the vasodilating components to the cation exchange resin was fairly strong, and the operation to remove hydrochloric acid and solvent from a large amount of the eluent generated by repeated operation of the hydrochloric acid elution in order to recover a sufficient amount of vasodilating components was complicated. Therefore, we considered that the separation utilizing a cation-π interaction between positive charge of the vasodilating components and phenyl groups having π electrons may be effective, and conducted a solid phase extraction using a phenyl group-bound carrier, and succeeded in separating the fractions in which vasodilating components alone have been concentrated.

We considered that the reason for this is because vasodilating substances form a particularly stronger cation-π interaction compared to other co-existing compounds; we thus examined HPLC separation conditions using a column (PFP column) filled with a carrier to which pentafluorophenyl groups, that were expected to show stronger cation-π interaction, were bonded, and succeeded in further concentrating and separating vasodilating components.

Based on the above findings, we have established a method of producing a fermented food extract of the present invention.

That is, for example, a fermented food extract composition showing blood pressure-lowering effect and vasodilatory effect upon oral administration is produced by the following steps: a step wherein a lyophilizate or concentrate of fermented buckwheat is suspended in acetone, stirred by shaking, then centrifuged to obtain a supernatant, and the supernatant is vacuum concentrated (step 1),
a step wherein said vacuum concentrate is eluted using a solid phase extraction cartridge filled with a carrier to which a phenyl group is bonded, with acid-containing water as an eluent (step 2),
then, a step wherein said eluate is fractionated and purified using a column (PFP column) filled with a carrier to which a pentafluorophenyl group is bonded, with acidic methanol-containing water as a mobile phase (step 3).

The fermented buckwheat used in the production method of the present invention can be produced according to the production method of JP A 2012-162503 (Patent Document 3). Specifically buckwheat sprouts from which cultivation bed and hulls of seeds have been removed are immersed in 100-ppm sodium hypochlorite for 10 min for sterilization, cut and pulverized, and the resulting pulverized product is placed in a sealed container, to which a lactic acid bacteria starter (Lactobacillus plantarum) is added at 25 mL per 1.0 kg of the pulverized product; then, after nitrogen replacement, the resulting sample is stationary fermented at room temperature for 6 days, filtered or centrifuged to obtain fermented buckwheat as a reddish brown liquid, which is then lyophilized or vacuum concentrated to obtain a lyophilizate or a vacuum concentrate of the fermented buckwheat.

Examples of solid phase extraction cartridge used in step 2 include a sol id phase extraction cartridge (HyperSep Phenyl SPE Columns, Shimadzu STRATA Phenyl, Agilent Bond Elut PH) and others, and a solid phase extraction cartridge (GI Science InertSep®) is most preferred. Examples of acid-containing water used as an eluent include hydrochloric acid-containing water, acetic acid-containing water, trifluoroacetic acid (TFA)-containing water, and phosphoric acid-containing water or the like; and formic acid-containing water is preferred, and 0.01% formic acid-containing water is most preferred.

Examples of reverse phase column used in step 3 include a phenyl column (Imtakt Unison UK-Phenyl, GL Science InertSustain Phenyl, GL Science Inertsil Ph-3, COSMOSIL 5PFP Packed Column) and the like, and PFP column (YMC-Triart PFP) is most preferred. Examples of acidic methanol-containing water used as a mobile phase include water containing hydrochloric acid-methanol, water containing acetic acid-methanol, water containing TFA-methanol, and water containing phosphoric acid-methanol; and water containing formic acid-methanol is preferred, and water containing 0.01% formic acid-33% methanol is mpst preferred.

Vasodilating components of the fermented buckwheat were separated by the above-described method of fractionation and purification; as a result, acetylcholine, propionylcholine, and butyrylcholine could be isolated with high purity, and their structure was determined by NMR and mass spectrometry.

Moreover, by LC-MS/MS analysis using PFP column, in addition to the above 3 kinds of choline, we have identified lactylcholine, levulinylcholine, carnitine and its methyl ester, trimethylglycine (betaine) and its methyl ester, ethyl ester and propionyl ester, and phosphocholine.

This finding is the first case in which lactylcholine has been identified as a natural product; furthermore, levulinylcholine isolated by the method of fractionation and purification of the present invention has not yet been reported or suggested.

Using hydrochloride salts of acetylcholine, propionylcholine, and butyrylcholine isolated and purified by the method of fractionation and purification of the present invention, an isometric tension test of blood vessels using thoracic aorta specimens of SHRs, and a single oral administration test to SHRs were carried out.

In the isometric tension test of blood vessels, acetylcholine showed a vasodilatory effect from 10⁻⁹ M, and the maximum at ion rate was 89.9% at 10⁻⁶ M, and EC₅₀ was 2.72×10⁻⁸

Propionylcholine showed a dilation reaction from 10⁻⁷ M, and the maximum dilation rate was 95.2% at 10⁻⁴ N, and EC₅₀ was 2.27×10⁻⁶ M.

Butyrylcholine showed a weak vasodilatory effect of 7.14% dilation at the addition of 10⁻⁴ M.

Single oral administration test was carried out with a dose of 10⁻¹¹ mol/kg, with consideration given to the single oral test results and the content in the fermented buckwheat.

As a result, in the acetylcholine administration group and propionylcholine administration group, a significant vasodilatory effect as compared to the control group was induced at 9 h after administration.

This result overturns the established theory that acetylcholine does not cause a physiological activity by oral administration.

Moreover, the finding that propionylcholine causes a physiological activity by oral administration has not yet been reported or suggested.

In the fermented buckwheat, it is speculated that other quaternary alkylammonium compounds (butyrylcholine, lactylcholine, levulinylcholine, phosphocholine, carnitine and its methyl ester, trimethylglycine (betaine) and it is methyl ester, ethyl ester and propionyl ester) are present in a mixed state, inducing excellent vasodilatory effect and blood pressure-lowering effect; therefore, even if these compounds alone have only weak vasodilatory effect and blood pressure-lowering effect, it is considered that these compounds together with acetylcholine and propionylcholine synergistically contribute to the blood pressure-lowering effect.

In addition, organic acids contained in the fermented buckwheat (lactic acid, acetic acid, citric acid) contribute in stabilizing choline esters such as acetylcholine and propionylcholine by maintaining the acidity of the solution, thereby inducing the effects by oral administration; therefore, known hypotensive components such as amino acids (tyrosine, GABA) are also considered to contribute to the blood pressure-lowering effect together with acetylcholine and propionylcholine.

Examples of raw materials for the fermented food of the present invention are not particularly limited as long as they are edible, and include stems, leaves and seeds (grains, beans) from plants, and animal-derived milk. Bacteria used for fermentation of the fermented food of the present invention are not particularly limited, and may be any kinds as long as they are not pathogenic.

The present inventors have examined for the fermented food other than fermented buckwheat in terms of similar fractionation and purification, and found that the following substances are contained in various fermented foods such as yogurt, black vinegar, natto and Japanese sake: acetylcholine, propionylcholine, lactylcholine, phosphocholine, carnitine, trimethylglycine and its methyl ester and ethyl ester.

In particular, we have confirmed that a fairly high content propionylcholine is present in the above fermented foods. This finding is a new one that has not yet been reported or suggested.

In addition, we have found that black vinegar comprises butyrylcholine, lactylcholine, phosphocholine, carnitine, trimethylglycine (betaine) and its methyl ester and ethyl ester; yogurt comprises lactylcholine, phosphocholine, carnitine and its methyl ester, trimethylglycine (betaine) and its methyl ester, ethyl ester and propionyl ester; natto comprises butyrylcholine, lactylcholine, phosphocholine, carnitine and its methyl ester, trimethylglycine (betaine) and its methyl ester, ethyl ester and propionyl ester; Japanese sake comprises butyrylcholine, lactylcholine, phosphocholine, carnitine and its methyl ester, trimethylglycine (betaine) and its methyl ester and ethyl ester.

From this finding, we can speculate that at least in the above fermented foods such as yogurt, black vinegar, natto and Japanese sake, it is possible to obtain extract compositions which exhibit similar activities with the fermented buckwheat extract composition, by performing fractionation and purification of active ingredients in accordance with the production method of the fermented buckwheat extract composition of the present invention.

The extract compositions of the present invention can be used as an active ingredient of various functional health foods or pharmaceutical compositions.

In the case of food, they can be used as a food composition, in combination with suitable food additives. Other than food compositions, they can be provided in a routinely ingestible form, for example, used as beverages by blending in green tea, black tea, oolong tea, and zakkoku-cha (tea made from various cereals), etc., and as food products by blending in biscuits, breads, candies, etc. Furthermore, it is also possible to use them in so-called supplements, in a suitable dosage form according to the formulation of pharmaceuticals below.

When a pharmaceutical is intended, the present extract composition in combination with suitable pharmaceutical additives can he used with various dosage forms according to general procedure of preparation of pharmaceuticals. Examples of such dosage forms include oral administration preparations including solid preparations such as powders, granules, capsules, pills and tablets, and liquid preparations such as solutions, suspensions and emulsions.

When the extract of the present invention is used as a food, examples of usage include not only general foods and drinks, but also functional health foods to promote health by exerting specific functions.

Examples of specific forms in such cases include supplements consisting of capsules, tablets, powders, and granules, etc., bakery foods such as breads, cakes and cookies, etc., seasonings such as source, soup, dressing, mayonnaise, etc., dairy products such as milk, yogurt, cream, etc., confectionery such as chocolates, candies, etc., or a variety of beverages such as green tea, black tea, oolong tea, barley tea, zakkoku-cha, juice, vegetables, milk beverages, soft drinks and carbonated beverages, etc., all containing the composition of the present invention as an active ingredient.

When the extract composition of the present invention is used as an active ingredient of a pharmaceutical composition, its dose differs depending on the ratio of each component, and on various factors such as patient's age, weight, sex, symptoms, and administration method; in the case of adult with oral administration, the dose per day can be selected from the range of generally 0.1 mg-100 g, and in the case of parenteral administration, it is selected from the range of generally 0.1 mg-1000 mg. The dose can be increased or decreased as appropriate depending on the degree of symptom improvement. Regarding the frequency of administration, it can be administered in one to several times a day.

When the extract composition of the present invention is used as a food, its amount of intake can be selected in accordance with the above case of pharmaceuticals with oral administration. However, in the case of food and drink, since the amount and frequency of the dose are not particularly limited which is different from pharmaceuticals, the amount of intake may be selected without being limited to the above range as long as there is no particularly severe symptoms, taking into consideration the purpose of maintaining health, as well as taste and palatability.

### EXAMPLES

Hereinafter, embodiments of the present invention will be described by way of examples and test examples; however, the present invention is not limited to the following examples.

### Example 1

### Production of Fermented Buckwheat

Fermented buckwheat was produced in accordance with the production method of JP A 2012-162503 (Patent Document 3).

As the buckwheat sprouts, those grown for about 10 days from sowing and harvested were purchased from Salad Cosmo Co., Ltd. The buckwheat sprouts from which cultivation bed and hulls of seeds had been removed (10 kg) were immersed in 100-ppm sodium hypochlorite (20 L) for 10 min for sterilization, cut into a length of about 2.0 cm, and finely pulverized.

The obtained pulverized product was placed in a sealed container (10 L), to which a lactic acid bacteria starter (Lactobacillus plantarum) was added at 25 mL per 1.0 kg of the pulverized product; then, after nitrogen replacement, the resulting sample was stationary fermented at room temperature for 6 days, filtered or centrifuged to obtain fermented buckwheat (6.8 kg) as a reddish brown liquid. This was then lyophilized or vacuum concentrated in accordance with conventional method to obtain a lyophilizate or a vacuum concentrate of the fermented buckwheat, which was subjected to isolation and purification of vasodilating components.

### Example 2

### Fractionation of Vasodilating Components of Fermented Buckwheat

### Test Example 1

### Examination of Purification Conditions of Vasodilating Components

First, using the lyophilizate obtained from 2 kg of the fermented buckwheat, we revealed chemical properties of vasodilating substances of the fermented buckwheat and examined the purification conditions. After centrifuging the fermented buckwheat (2 kg) (3220 g, 30 min, 4° C.), residues were removed and the resulting supernatant was lyophilized to obtain a deep red and hygroscopic lyophilizate of the fermented buckwheat (2.05% yield).

In the examination of each condition, concentration of the active ingredient was checked by identifying fractions showing vasodilatory effect by measurement test of isometric tension in blood vessels at each purification stage.

### pH Stability Test

Fermented buckwheat (liquid) used in this examination of conditions was acidic with pH 3.6, and showed a vasodilatory effect from 0.50 μg/mL for the blood vessel specimens of thoracic aorta of 12-week-old male SHRs, with EC₅₀ of 8.30 μg/mL.

To test pH stability of the fermented buckwheat, a lyophilizate of the fermented buckwheat (10 mg) was dissolved in purified water, and adjusted to pH 7.0, pH 9.0, and pH 12.0 by the addition of 1N sodium hydroxide, to which purified water was added to make a volume of 1 mL. A lyophilizate of the fermented buckwheat (10 mg) dissolved in purified water (1 mL) was used as a control sample (pH 4.0). After being left stand for 0.5, 1, 3, 6, 12, and 24 h at room temperature, vasodilatory effect of each solution with a fermented buckwheat concentration of 50 μg/mL was confirmed by a measurement test of isometric tension in blood vessels. As a result, while the vasodilatory effect was maintained for the control sample and the sample with pH 7.0 even at 24 h after, the vasodilatory effect was lost for the sample with pH 9.0 at 6 h after, and for the sample with. pH 12.0 at 0.5 h after.

### Organic Solvent Extraction Test

A lyophilizate of the fermented buckwheat (100 mg) was suspended in 1 mL each of ethanol, acetone, and ethyl acetate, and the samples were shaken in a shaking incubator (1811 g, 6 h, room temperature), then centrifuged (3220 g, 30 min, 4° C.) to separate the supernatant and precipitate.

The precipitate was again suspended in each of the organic solvents, and similarly separated into supernatant and precipitate by centrifugation. Combined supernatant was vacuum concentrated, from which the solvent was completely removed by a vacuum pump.

The yield of each of the organic solvent extract was 975 μg (0.98%) for ethanol extract, 512 μg (0.51%) for acetone extract, and 210 μg (0.21%) for ethyl acetate extract.

In the measurement test of isometric tension in blood vessels, vasodilatory effect of each solution at the extract concentration of 50 μg/mL was confirmed. As a result, vasodilation rates by ethanol extract, acetone extract, and ethyl acetate extract were 22%, 49% and 0%, respectively.

### Gel Filtration Chromatography

A lyophilizate of the fermented buckwheat (1.25 g) was dissolved in pure water (10 mL), and injected into an open column (4.2 cm×50 cm) filled with Sephadex G-15 (30 g) with fractionation molecular weight of 700-1500. After removal of 300 mL of void volume, 50 mL each was fractionated to obtain gel filtration fractions 1-8. Each of the fractionated fractions was lyophilized, and the lyophilizate was subjected to a measurement test of isometric tension in blood vessels. The amount and yield of each fractionated fraction by gel filtration chromatography are shown in Table 1.

TABLE 1
Yield of gel filtration fractions

| | Fraction | | | | | | | | |
|---|---|---|---|---|---|---|---|---|---|
| | 1 | 2 | 3 | 4 | 5 | 6 | 7 | 8 | Total |
| Amount (mg) | 77.2 | 109.0 | 160.1 | 238.0 | 192.9 | 136.1 | 116.2 | 36.2 | 1103.3 |
| Yield (%) | 6.2 | 8.7 | 12.8 | 19.0 | 15.4 | 10.9 | 9.3 | 2.9 | 88.3 |

Vasodilatory effect in 25 ρg/mL of each fraction was confirmed in the measurement test of isometric tension in blood vessels, and the vasodilatory effect was observed in the gel filtration fractions 3 and 4. As a result of examination of the vasodilatory effect of the gel filtration fractions 3-5 by cumulative addition, the gel filtration fraction 3 showed a dilation rate of 4.80% by the addition of 1.0 ng/mL, confirming a dose-dependent vasodilation reaction, and the maximum dilation rate was 61.9% at the addition of 5.0 μg/mL. The gel filtration fraction 4 showed a dilation rate of 2.74% by the addition of 1.0 ng/mL, and the maximum dilation rate was 71.2% at the addition of 5.0 μg/mL. The gel filtration fractions 3 and 4 showed a start of vasodilation at a concentration as low as 1/500 of that of the fermented buckwheat, indicating that they contain a substance exhibiting strong vasodilatory effect. On the other hand, gel filtration fraction 5 showed a vasoconstriction reaction in a dose-dependent manner, and thus the fermented buckwheat also contained vasoconstriction components in addition to vasodilating components. It was considered that by the gel filtration chromatography, the vasoconstriction components contained in the gel filtration fraction 5 were removed and vasodilating substances were concentrated.

### Ion Exchange Treatment

### Strong-Acid Cation Exchange Resin Treatment

A lyophilizate of the fermented buckwheat (2000 mg) was dissolved in pure water (5 mL), and the pH was adjusted to 7 with 1N NaOH, then the sample was centrifuged (3220 g, 4° C., 20 min), and supernatant was collected and diluted j n a measuring cylinder to a total of 10 mL. The obtained supernatant (2.5 mL) was added to a suspension of proton type strong-acid cation exchange resin (Amberlite IR120B, 5 mL) and purified water (2 mL), treated at room temperature for 2 h with gentle stirring, and filtered to separate the treated solution and the resin. The resin was washed twice with pure water (15 mL), and combined with the treated solution. 2N HCl (30 mL) was added to the washed resin, treated at room temperature for 30 min with gentle stirring to elute adsorbed components, and filtered to separate the hydrochloric acid eluent and the resin. The resulting treated solution was treated with ion exchange resin, and this operation was repeated twice; all of the resulting eluent in each operation were combined. The treated solution (99 mL) and hydrochloric acid-eluent (90 mL) were each diluted in a measuring cylinder to a total of 300 mL, subjected to 4-fold dilution, then vasodilatory effect was confirmed by a measurement test of isometric tension in blood vessels. As a result, vasodilatory effect was observed in the hydrochloric acid elution fractions; it was 56.0% when. 100 μL of the hydrochloric acid elution fraction were added. Assuming that all of the vasodilating components in the fermented buckwheat are adsorbed to the ion exchange resin, the concentration of the fermented buckwheat at this time corresponds to 10 μg/mL. No vasodilatory effect was observed in the treated solution.

### Weak-Acid Cation Exchange Resin Treatment

A lyophilizate of the fermented buckwheat (2000 mg) was dissolved in pure water (5 mL), and the pH was adjusted to 7 with 1N NaOH, then the sample was centrifuged (3220 g, 4° C., 20 min), and supernatant was collected and diluted in a measuring cylinder to a total of 10 mL. The obtained supernatant (2.5 mL) was added to a suspension of sodium type weak-acid cation exchange resin (Amberlite IRC76, 5 mL) and purified water (2 mL), treated at room tern for 2 h with gentle stirring, and filtered to separate the treated solution and the resin. The resin was washed twice with pure water (15 mL), and combined with the treated solution. 2N HCl (30 mL) was added to the washed resin, treated at room temperature for 30 min with gentle stirring to elute the adsorbed component, and the resulting solution was filtered to separate the hydrochloric acid eluent and the resin. The resulting treated solution was treated with ion exchange resin, and this operation was repeated twice; all of the resulting elution in each operation were combined. The treated solution (94 mL) and the hydrochloric acid eluent (90 mL)) were each diluted in a measuring cylinder to a total of 300 mL, subjected to 4-fold dilution, then vasodilatory effect was confirmed by a measurement test of isometric tension in blood vessels. As a result, vasodilatory effect was observed in the treated solution and in the hydrochloric acid elution fractions; it was 7.3% when. 100 μL of the treated solution were added, and 23.3% when 100 μL of the hydrochloric acid elution fraction were added. Adsorption of the vasodilating components of fermented buckwheat to the weakly-acid cation exchange resin was considered to be insufficient.

### Solid Phase Extraction

A solid phase extraction cartridge (GL Science InertSep® PH, 5 g) was activated with methanol (30 mL), washed with 0.01% formic acid-containing water (30 ml), and equilibrated with pure water (30 mL), then an acetone extract of fermented buckwheat (1.0 g) dissolved in pure water (2.5 mL) was applied to the cartridge; elution was carried out with pure water (30 mL) and 0.01% formic acid-containing water (30 mL). Elution fractions by pure water and elution fractions by 0.01% formic acid-containing water were lyophilized, and the vasodilatory effect was confirmed by a measurement test of isometric tension in blood vessels. As a result, vasodilatory effect was observed in the elution fractions by 0.01% formic acid-containing water, and the vasodilation rate was 35.6% at 0.5 μg/mL. No vasodilatory effect. was observed in the elution fractions by pure water.

### HPLC Separation Conditions

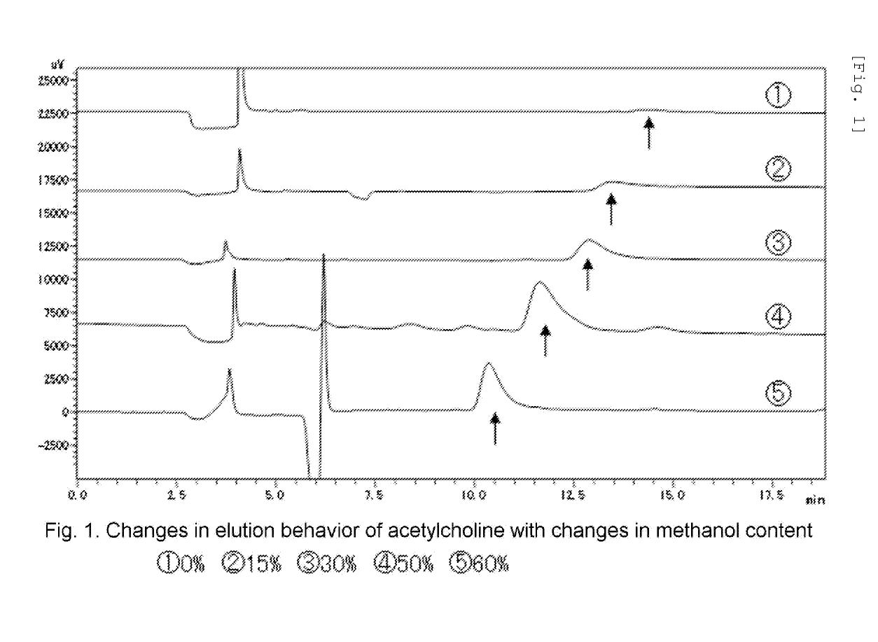

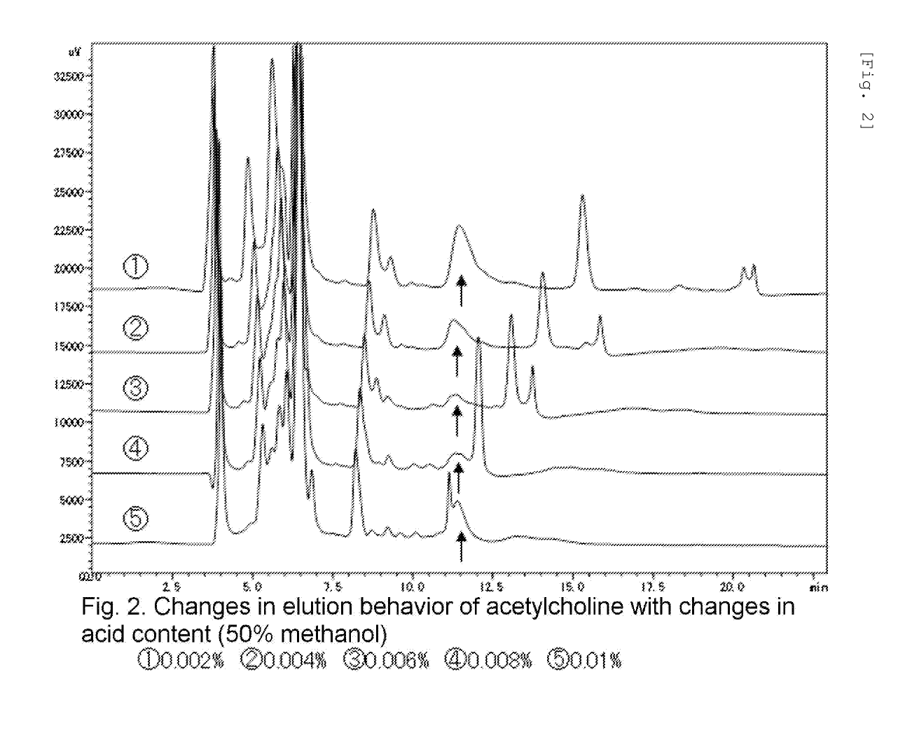

Using a reversed phase column (YMC-Triart PFP, 5 μm, 4.6 mm×250 mm) to which a pentafluorophenyl group is bonded, we investigated separation conditions of vasodilating components in the fermented buckwheat sol id phase extract (1 mg/mL). First, using an acetylcholine preparation which is a candidate of vasodilating component, conditions were studied by changing methanol content and acid content of the eluent. Namely, 60 μL of acetylcholine preparation (0.1 mg/ml) were injected at a flow rate of 0.5 mL/min, and 0, 5, 15, 30, 50, and 60% methanol-containing water and 0.002, 0.004, 0.006, 0.008, 0.01, and 0.015% formic acid-containing 50% methanol were used as an eluent, changes in the elution behavior of acetylcholine were examined at a separation temperature of 30° C. As a result, as the methanol content increased, elution time of acetylcholine was accelerated and the peak shapes became good. In addition, although the formic acid content did not have much effect on the retention time of acetylcholine, as the acid content increased, the detection peaks were reduced by an increase in the background. From the above results, it was found that the water containing 0.01% formic acid-50% methanol s most suitable for the analysis of acetylcholine. FIG. 1 shows changes in the elution behavior of acetylcholine with changes in the methanol content, and FIG. 2 shows changes in the elution behavior of acetylcholine with changes in the acid content.

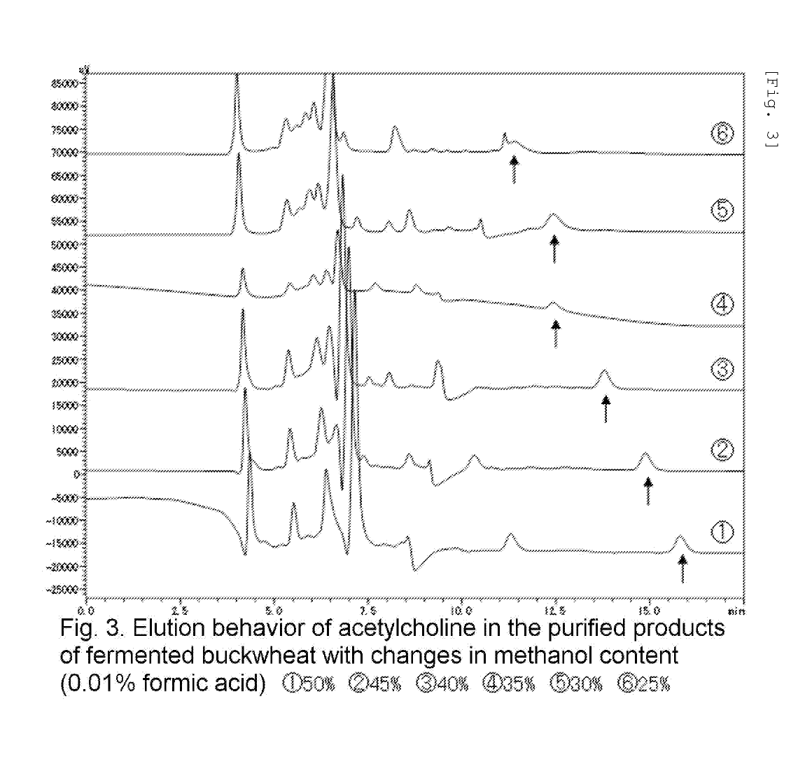

Next, separation conditions of solid phase extracts of fermented buckwheat were investigated. Based on the results of the analysis conditions of acetylcholine, HPLC analysis was performed with 0.01% formic acid by changing the methanol content at 25, 30, 35, 40, 45, and. 50%. As a result, separation by the methanol content of 35% was good. FIG. 3 shows the behavior of purified products of fermented buckwheat with changes in the methanol content under fixed condition of acid content (0.01%).

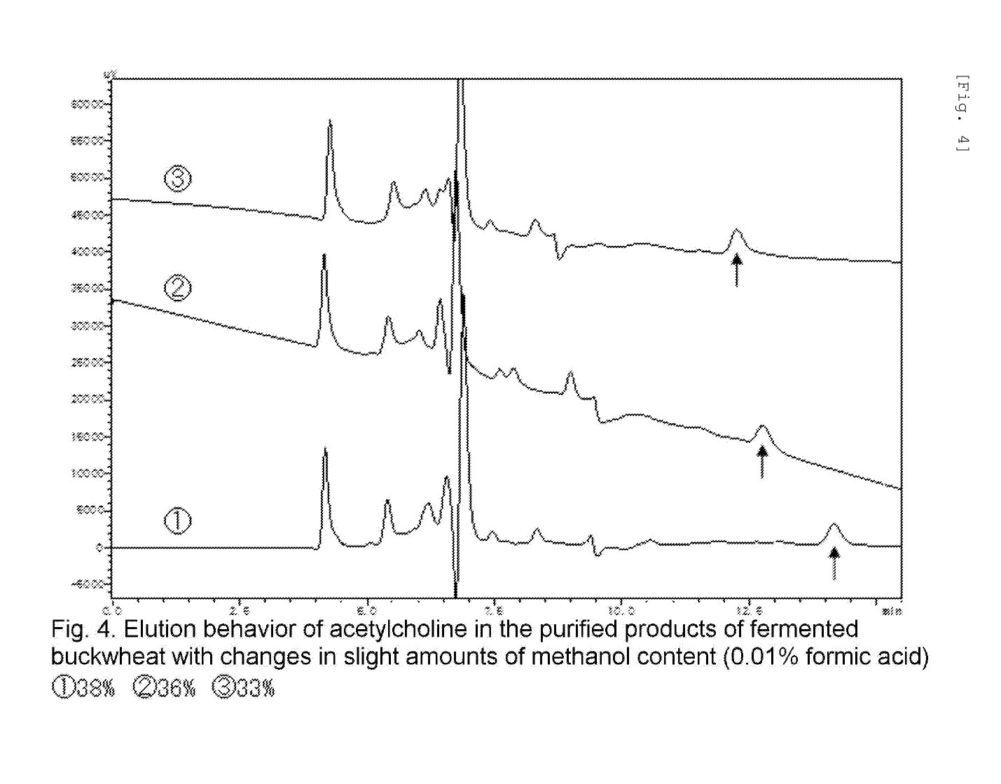

Furthermore, when the methanol content was finely changed (38, 36, 33%), best separation of the second half of the chromatogram was obtained with 33% methanol FIG. 4 shows the behavior of purified products of fermented buckwheat with changes in the methanol content under fixed condition of acid content (0.01%).

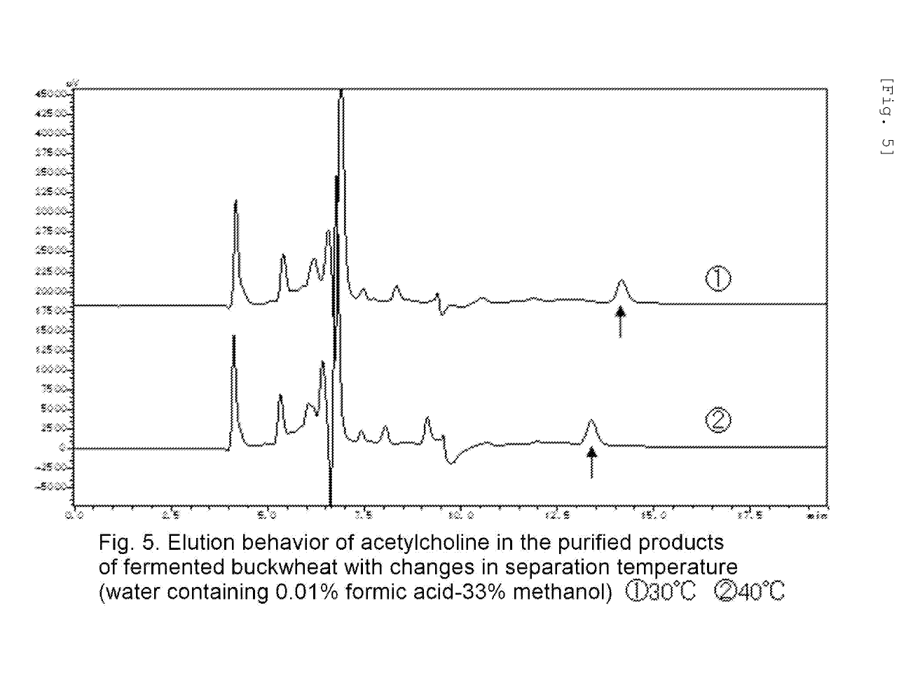

Finally, separation with the water containing 0.01% formic acid-33% methanol was examined by setting the separation temperature at 30° C. and 40° C.; better separation was obtained at 40° C. FIG. 5 shows changes in the chromatogram due to changes in the separation temperature.

From the above results, optimum separation conditions were determined as follows
Column: YMC-Triart® PPP, 5 μm, 4.6 mm×250 mm
Injection volume: solid phase extract of fermented buckwheat (0.1 mg/mL) 60 μL
Mobile phase: water containing 0.01% formic acid-33% methanol (Isocratic)
Flow rate: 0.5 mL/min
Temperature: 40° C.

### Example 3

### Purification of Vasodilating Components of Fermented Buckwheat

100 kg of fermented buckwheat were vacuum concentrated, and subjected to acetone extraction, solid phase extraction, gel filtration chromatography, and HPLC to purify vasodilating components of the fermented buckwheat.

### Acetone Extraction of Vasodilating Components of Fermented Buckwheat

The fermented buckwheat was centrifuged (3220 g, 30 min, 4° C.), and the residue was removed after centrifugation. The resulting supernatant was concentrated using a rotary evaporator under reduced pressure to obtain a concentrate of the fermented buckwheat. The concentrate of the fermented buckwheat (approximately 120 g) and acetone (300 mL) were added in a 2-L mortar, and vasodilating components were extracted by grinding the sample with a pestle for 5 min, and the supernatant was collected by an aspirator. Acetone (300 mL) was further added to the residue, and after sufficient grinding for 5 min with a pestle, the supernatant was collected similarly. This operation was repeated a total of 12 times. The resulting acetone solution was concentrated using a rotary evaporator under reduced pressure to obtain an acetone crude extract. The acetone crude extract (approximately 60 g) and ethyl acetate (approximately 200 mL) were added to a 500-mL eggplant-shaped flask, and after stirring well with a glass rod for 5 min, the supernatant was removed after 10 min of ice cooling. Ethyl acetate (approximately 200 mL) was added again to the residue, stirred well for 5 min with a glass rod, and the supernatant was removed in a similar manner. This operation was repeated a total or 12 times, then the resulting residue was dried under reduced pressure for more than 3 h in a desiccator to obtain an acetone extract.

### Solid-Phase Extraction and Purification of Vasodilating Components of Fermented Buckwheat and Fractionation by Gel Filtration Chromatography

As a pre-treatment of gel filtration chromatography, a solid phase extraction cartridge (GL Science InertSep® PH, 50 g) was activated with methanol (300 mL), washed with 0.01% formic acid-containing water (300 mL), and equilibrated with pure water (300 mL); then an acetone extract of the fermented buckwheat (10 g) was dissolved in pure water (20 mL) and applied to the cartridge; after elution of contaminants by pure water (300 mL), vasodilating components were eluted with water containing 0.01% formic acid (300 mL). The resulting elution was concentrated using a rotary evaporator under reduced pressure to obtain a solid phase extract.

Subsequently, in the gel filtration chromatography, a column (4.2 cm×50 cm) was filled with Sephadex G-15 (230 g), and the solid phase extract (2 mL) prepared in 500 mg/mL of pure water was injected, eluted with pure water to obtain 410-485 mL of fractionated elution. The fractionated elution was vacuum concentrated and lyophilized to obtain a gel filtration product.

### LC-MS and LC-MS/MS Analyses of Vasodilating Components of Fermented Buckwheat

LC-MS analysis and LC-MS/MS analysis of gel filtration products were carried out. The gel filtration product and an analysis solvent were added in a 1.5-mL volume microtube to prepare a sample solution of 1 mg/mL concentration, which was filtered through Millex®-LH filter (0.45 μm) to obtain a LC-MS analytical sample. As the column, a reverse phase column YMC-Triart® PFP (5 μm, 4.6 mm×250 mm) to which PFP groups were bonded was used, and water containing 0.01% formic acid-33% methanol was used as the mobile phase. Ionization mode EST⁺, flow rate: 0.5 mL/min (LC) and 0.3 mL/min (MS), separation temperature: 40° C., injection volume: 10 μL, capillary voltage: 3500 V, N₂ gas flow (cone): 50 L/h, N₂ source temperature: 120° C., N₂ desolvation temperature: 350° C. In addition, the ionization voltage was examined at 10-60 V for each peak to be analyzed, and the condition of the most superior ionization, i.e, cone voltage of 10 V and collision voltage of 30 V, was used. For major ion peaks observed by LC-MS analysis, a neutral loss scan was carried out to identify those having a trimethylammonium group (mass-to-charge ratio of 59) as a quaternary alkylammonium compound. Then, LC-MS/MS analysis was carried out using the mass-to-charge ratio or the identified quaternary alkylammonium compound as a precursor ion. As a result, as the quaternary alkylammonium compounds, the following substances were identified: acetylcholine, propionylcholine, butyrylcholine, lactylcholine, levulinylcholine, carnitine and methyl ester, trimethylglycine (betaine) and its methyl ester, ethyl ester and propionyl ester, and phosphocholine.

### Example 4

### Isolation and Purification of Acetylcholine, Propionylcholine, and Butyrylcholine

### HPLC Purification of Vasodilating Components of Fermented Buckwheat

Referring to the above LC-MS analysis results, vasodilating components contained in the gel filtration product were purified by HPLC to isolate the vasodilating components. The gel filtration product and HPLC analysis solvent were added to a 1.5-mL volume microtube to prepare a sample solution of 100 mg/mL concentration, which was filtered through Millex®-LH filter (0.45 μm) to obtain a separation sample. Prominence HPLC system was used as the analytical system, and YMC-Friary® PFP (5 μm, 20 mm×250 mm) was used as the column. The analysis conditions are as follows: mobile phase: water containing:0.01% formic acid-33% methanol, flow rate: 5.671 mL/min, separation temperature: 40° C., detection wavelength: 215 nm, injection volume: 50 μL of the gel filtration product. Fractions containing vasodilating components were fractionated, and the same purification was repeated if necessary, to obtain 3 kinds of pure components. After addition of hydrochloric acid, the final purified product was vacuum concentrated. and lyophilized to obtain a hydrochloride the purified product, which was used for structure determination and testing.

### NMR Analysis of Vasodilating Components of Fermented Buckwheat

NMR analysis was carried out for the 3 kinds of HPLC purified products. Regarding the obtained fractionated products, each of the lyophilizate (1.6 mg) was dissolved by addition of heavy water (D₂O, 0.16 ml) to prepare NMR samples. NMR apparatus with ¹H resonance frequency of 500 MHz (ADVANCE DRX500, Bruker Biospin Co., Ltd., Yokohama) was used, with a cumulative number of 128 times. As a result, NMR charts of the 3 kinds of HPLC purified products were identical to those of commercially available special-grade acetylcholine hydrochloride, synthesized propionylcholine and butyrylcholine; accordingly, they were identified as acetylcholine, propionylcholine, and butyrylcholine.

### Mass Analysis of Vasodilating Components of Fermented Buckwheat

MALDI-TOF/MS analysis was carried out: for the 3 kinds of HPLC purified products. Regarding the obtained fractionated products, each of the lyophilizate (0.01 mg) was dissolved in 50% acetonitrile containing 0.1% IFA (15 μL) to prepare a sample for measurement. 1 μL each of the sample and a 0.1% TFA-containing 50% acetonitrile solution of 2,5-dihydroxybenzoic acid as a matrix (7500, 1500, 750 μg/mL) were mixed on a plate and dried in a desiccator. In the MALDI-TOF MS analysis, a MALDI-TOF MS mass spectrometer (AB SCTEX TOF/TOF 5800, AB Sciez Japan Ltd., Tokyo) was used with a reflector mode, scan range of 10-300 m/z, and laser intensity of 5500 eV. As a result, 146.1182 m/z (theoretical value: 146.1176), 160.1337 m/z (theoretical value: 160.1332), and 174.1497 m/z (theoretical value: 174.1489) were observed, respectively, which correspond to the accurate mass of acetylcholine, propionylcholine, and butyrylcholine.

### Characterization of Vasodilating Components of Fermented Buckwheat

### Synthesis of Standard Substance

As a standard substance of each hydrochloride salt of the isolated and purified acetylcholine, propionylcholine and butyrylcholine, commercially available special-grade acetylcholine hydrochloride was used for acetylcholine, and those that have been synthesized, PPLO-purified and lyophilized as a hydrochloride salt our laboratory were used for propionylcholine and butyrylcholine. Propionylcholine and butyrylcholine can be synthesized in accordance with the following synthetic chart represented by the formulae (1) and (2)

### Synthesis of Propionylcholine

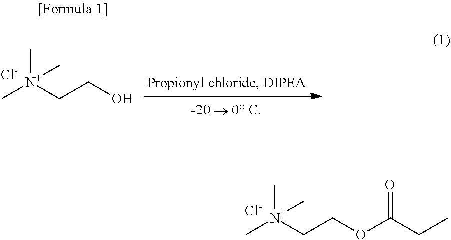

Synthesis of propionylcholine: Choline hydrochloride (0.42 g, 3.0 mmol) was dissolved in N,N-dimethylformamide (DMF, 6.0 mL), then propionyl chloride (0.56 g, 6.0 mmol) and diisopropylethylamine (DIPEA, 0.78 g, 4.5 mmol) were added and the sample was allowed to reach room temperature from 0° C. and react for 1 h. After completion of the reaction, DMF was distilled off, and a product was precipitated with dichloromethane (DCM)-ethyl acetate (1:1). The precipitate was recrystallized with DCM-ethyl acetate (5:1) and further recrystallized with DCM to obtain propionylcholine hydrochloride as a white powder. Yield: 92%, ¹H NMR (500 MHz, D₂O) δ (ppm): 0.96(3H, t, J=7.8 Hz, CH₃), 2.32(2H, q, J=7.5 Hz, CH₂), 3.08(9H, s, CH₃), 3.60(2H, t, J =2.5 Hz, CH₂), 4.42(2H, t, J=2.3 Hz, CH₂)
Synthesis of butyrylcholine

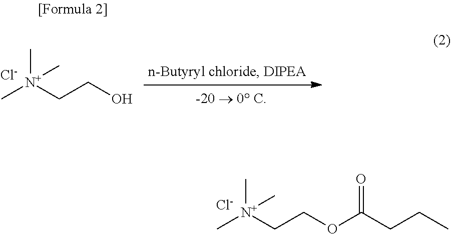

Synthesis of butyrylcholine: Choline hydrochloride (0.42 g, 3.0 mmol) was dissolved in DMF (6.0 mL) then butyryl chloride (0.62 g, 6.0 mmol) and DIPEA (0.78 q, 4.5 mmol) were added and the sample was al owed to reach room temperature from 0° C. and react for 1 h. After completion of the reaction, DMF was distilled off, and a product was precipitated with DCM-ethyl acetate (1:1). The precipitate was recrystallized with DCM-ethyl acetate 5:1) and further recrystallized with DCM to obtain butyrylcholine hydrochloride as a white powder. Yield: 89%, ¹H NMR (500 MHz, D₂O) δ (ppm): 0.78(3H, t, J=7.5 Hz, CH₃), 1.49(2H, 6, J=7.5 Hz, CH₂), 2.29 (2H, t, J=7.5 Hz, CH₂), 3.08(9H, s, CH₃), 3.60(2H, t, J=2.3 Hz, CH₂), 4.43(2H, t, J=2.3 Hz, CH₂)

### Laboratory Animals Used

Animal experiments were carried out according to the animal experiment guidelines of Shinshu University. In the present study, spontaneously hypertensive rats of 10-13 week-old males (SHR/NCrlCrlj, Charles River Laboratories Japan, Yokohama) were used as an experimental animal. SHRs were bred in individual cages in a breeding room controlled with a light-dark cycle of 1.2 h and a room temperature of 22-23° C. In acclimation breeding, rat standard diet (MF; Charles River Laboratories) and tap water were fed ad libitum. SHRs are a rat strain that has been isolated in 1963 by selection mating from Wistar-Kyoto rats that had been bred in the animal experimentation center of Kyoto University. They are referred to as spontaneously hypertensive rats or essential hypertensive rats; while systolic blood pressure of the separation maternal. Wistar-Kyoto rats is approximately 135 mmHg for mature males and approximately 132 mmHg for mature females, blood pressure of almost all the SHRs becomes 150 mmHg or higher at 2 months from the birth, and reaches almost a maximum value at 4-5 months, with many of the values showing 180-210 mmHg. In general, as a model animal of essential hypertension that s said to be account for approximately 90% of the hypertension in humans, SHRs have been used to elucidate pharmacological effects and food functions, and a mechanism of blood pressure rise.

### Measurement Test of Isometric Tension in Blood Vessels Using Substances Containing Vasodilating Components of Fermented Buckwheat

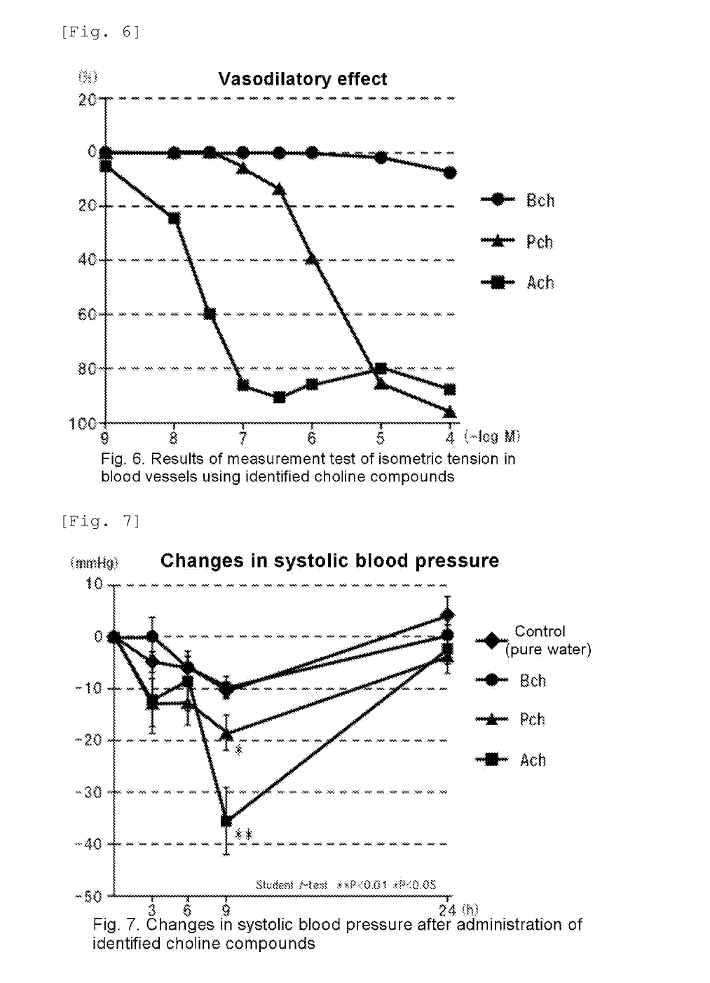

Thoracic aorta used for the test was removed from a male 10-12 week-old SHR; after removal of the connective tissue, it was cut into a length 2-3 mm to produce ring specimens. The resulting ring specimens were attached to an organ bus filled with Krebs solution of 37° C. (119 mM NaCl/4.7 mM KCl/1.1 mM KH₂PO₄/1.2 mM MgSO₄/25 mM NaHCO₃, pH 7.4) in which a mixed gas of 95% O₂-5% CO₂ was aerated, then a static tension of 1.5 g was applied (UFER UC-05A; Iwashiya Kishimoto Ika Sangyo K.K., Kyoto). Vascular tension was measured by UFER UM-203 transducer (Iwashiya Kishimoto Ika Sangyo). After 60-min incubation from the attachment, the ring specimens were constricted using a vasoconstrictor phenylephrine (final concentration of 0.30 μM). To the constricted ring specimens, commercially available acetylcholine, that is an endothelium-dependent vasodilator (final concentration of 100 μM) was added, and presence/absence of vascular endothelium was determined by presence/absence of vasodilatory effect. Then, the ring specimens were washed with Krebs solution to remove the drugs, incubated at static tension for 10 min, and again constricted by 0.30 μM phenylephrine. After this operation was repeated twice, 0.30 μM phenylephrine was added again to make sure that constriction has reached the maximum, then each sample dissolved in the Krebs solution was added in a cumulative manner such that its final concentration becomes 10 ⁻⁹, 10⁻⁸, 10^{-7.5}, 10⁻⁷, 10^{-6.5}, 10⁻⁶, 10⁻⁵, and 10⁻⁴ M. Vasodilatory effect (dilation rate, %) was represented using the vascular tension upon constriction by phenylephrine as a reference. Results of the isometric tension test in blood vessels for purified acetylcholine, propionylcholine, and butyrylcholine are shown in FIG. 6.

Purified acetylcholine showed a 1.52% dilation rate at the addition of 1.0⁻⁹ M, and thereafter showed a dose-dependent dilation reaction up to 10⁻⁶, then a slight constriction at higher concentrations. Maximum dilation rate was 89.9% at the addition of 10⁻⁶ M, confirming a large dilation at low dose. EC₅₀ was 2.72×10⁸ M. In addition, purified propionylcholine showed a dilation reaction from 10⁻⁷ M, and the maximum dilation rate was 95.2% at 10 ⁻⁴ M, and EC ₅₀ was 2.27×10⁻⁶ M. Meanwhile, purified butyrylcholine showed a dilation of 7.14% at the addition of 10⁻⁴ M, indicating weak dilatory effect compared to acetylcholine and propionylcholine. Commercially available acetylcholine and synthetic propionylcholine and butyrylcholine also showed a similar vasodilatory effect. The above results revealed that acetylcholine and propionylcholine isolated and purified from the fermented buckwheat have strong vasodilatory effects.

### Single Oral Administration Test of Substances Containing Vasodilating Components of Fermented Buckwheat

Single oral administration test was carried out using male 12-week-old SHRs with a body weight of 280-320 g. After acclimation breeding for 1 week, the rats were fasted for 12 h, and a sample was given to the rats by a single oral administration. The sample to be administered was dissolved in pure water, and given by a single oral administration with a dose of 10⁻¹¹ mol/kg (b.w.) to each 6 SHRs. Sample aqueous solutions of purified acetylcholine, propionylcholine and butyrylcholine were acidic, with a pH of 4.7, 4,3, and 4.4, respectively. To the control group, pure water was administered. Changes in the systolic blood pressure and diastolic blood pressure were measured using non-invasive blood pressure measuring instrument Softron BP-98A (Softron Co., Ltd., Tokyo) by tail cuff method, before administration and at 3, 6, 9, and 24 h after administration. Results of the single oral administration test of purified acetylcholine, propionylcholine and butyrylcholine were shown in FIG. 7.

As a result, in the acetylcholine administration group a significant systolic blood pressure-lowering effect was induced at 9 h after administration (p<0.01) compared to the control group, and the maximum vasodilatory effect at this time was 24.7 mmHg. In addition, in the propionylcholine administration group, a significant decrease in systolic blood pressure of maximum 8.05 mmHg (p<0.05) was observed at 9 h after administration. On the other hand, in the butyrylcholine administration group, no significant decrease in blood pressure was observed at 10⁻¹¹ mol/kg. Commercially available acetylcholine and synthetic propionylcholine and butyrylcholine also showed a similar blood pressure-lowering effect.

### Example 5

### Isolation and Purification of Lactylcholine and Levulinylcholine

According to the method described in Example 3, lactylcholine was identified as a natural product for the first time.

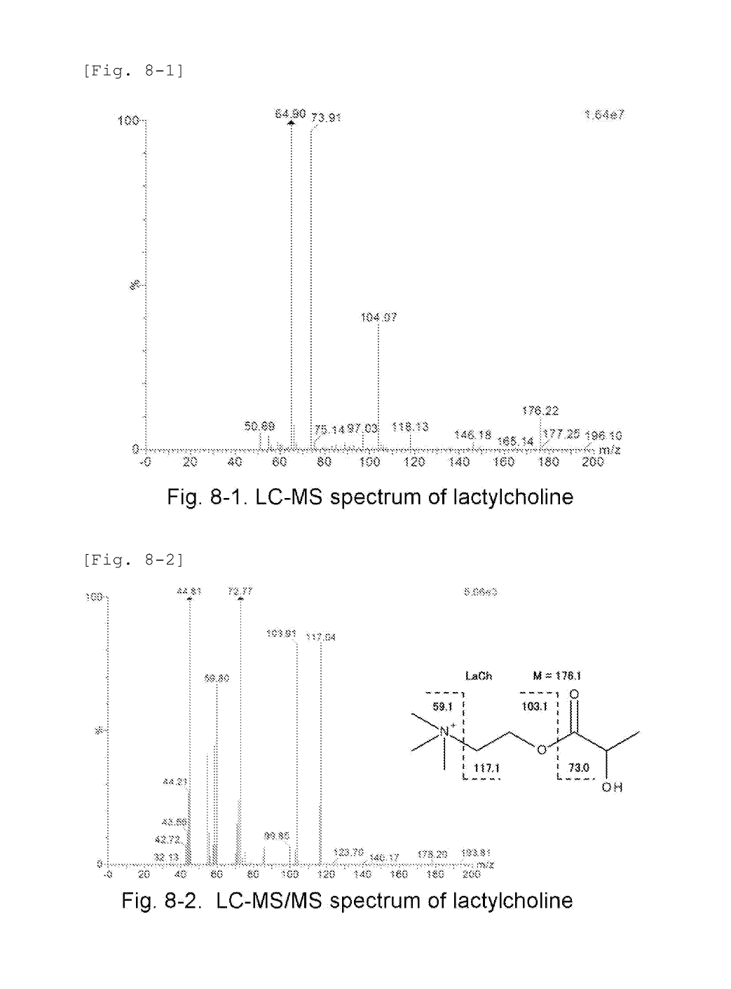

The mass spectrum of lactylcholine by LC-MS analysis was shown in FIG. 8-1, and that by LC-MS/MS analysis was shown in FIG. 8-2.

Furthermore, levulinylcholine represented by the formula (3) below, which is believed to be a novel compound, has been isolated and purified.

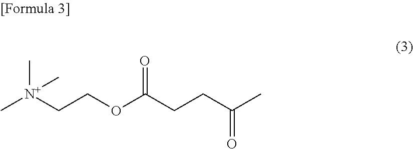

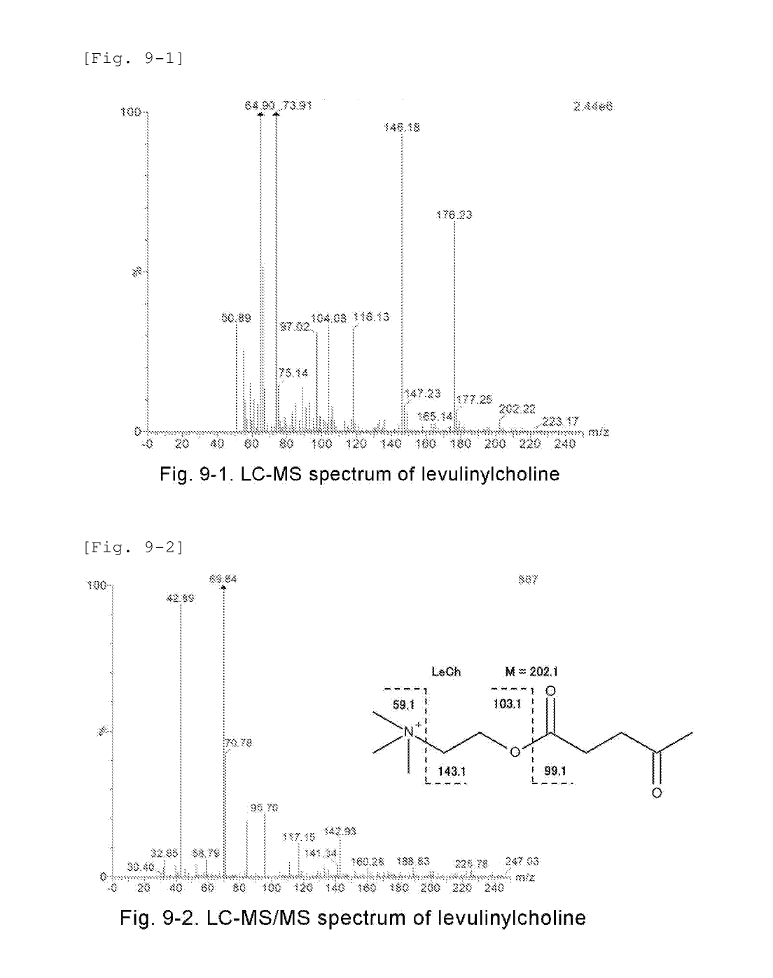

The mass spectrum of levulinylcholine by LC-MS analysis was shown in FIG. 9-1, and that by LC-MS/MS analysis was shown in FIG. 9-2.

### LC-MS/MS Analysis of Quaternary Alkylammonium Compounds in Fermented Buckwheat and in Other Fermented Foods

Quaternary alkylammonium compounds contained in fermented buckwheat, black vinegar, yogurt, and Japanese sake were subjected to LC-MS analysis. Each of the lyophilizates was weighed in a mortar (1 g of fermented buckwheat, 10 g each of black vinegar, yogurt, natto and Japanese sake) and 1 mg of carbachol was added as an internal standard. 30 mL of acetone were added and stirred for 3 min, and supernatant was collected into a 50-mL volume centrifugal tube and centrifuged (4° C., 1811 g, 10 min) to obtain a supernatant. 30 mL of acetone were added again to the residue and stirred, centrifuged, and supernatant was collected; this operation was repeated three more times to obtain an acetone extract. The acetone extract was vacuum concentrated, lyophilized and dissolved in pure water (2.5 mL), and subjected to solid phase extraction using a solid phase extraction cartridge (GL Science InertSep® PH, 5 g). In the sol id phase extraction, first, the cartridge was activated with methanol (30 mL), washed with 0.01% formic acid-containing water (30 mL), and equilibrated with pure water (30 mL), then the sample was added. Contaminants were removed with pure water (30 mL), and elution was performed with 0.01% formic acid-containing water (30 mL) to obtain a solid phase extract comprising quaternary alkylammonium compounds. The solid phase extract was vacuum concentrated, lyophilized, and dissolved in an analysis solvent (water containing 0.01% formic acid-33% methanol, 1 mL) and subjected to LC-MS analysis. Quantification of the compounds was carried out by specifying the mass-to-charge ratio of the quaternary alkylammonium compounds identified in the fermented buckwheat in a single ion monitoring (SIM) mode. The mass-to-charge ratios specified for the compounds as as follows: acetylcholine: 146.1, propionylcholine: 160.1, butyrylcholine: 174.2, lactylcholine: 176.1, levulinylcholine: 202.1, phosphocholine: 184.1, carnitine: 162.1, methyl carnitine: 176.1, trimethylglycine: 118.1, methyl trimethylglycine: 132.1, ethyl trimethylglycine: 146.1, propionyl trimethylglycine: 160.1. Other analysis conditions are the same as the above mentioned LC-MS analysis of vasodilating components of fermented buckwheat. Table 2 shows the results of quantification of the quaternary alkylammonium compounds. Contents of each compound were corrected in terms of material loss during purification process using quantification results of the internal standard, and expressed by an acetylcholine equivalent of the content of the quaternary alkylammonium compound in 100 g of fresh weight (100 g FW).

TABLE 2
Quantification results of quaternary alkylammonium
compounds in fermented food (μg/100 g FW acetylcholine equivalent)

| Quaternary | | | | | |
|---|---|---|---|---|---|
| alkylammonium | Fermented | Black | | | Japanese |
| compounds | buckwheat | vinegar | Yogurt | Natto | sake |
| Acetylcholine | 295.8 | 11.8 | 5.8 | 1.1 | 41.7 |
| Propionylcholine | 457.9 | 27.3 | 1576.5 | 95.2 | 134.0 |
| Butyrylcholine | 94.4 | 24.1 | N.D. | 66.4 | 16.8 |
| Lactylcholine | 37.3 | 10.7 | 10.5 | 37.5 | 7.5 |
| Levulinylcholine | 31.1 | N.D. | N.D. | N.D. | N.D. |
| Phosphocholine | 16.1 | 9.7 | 11.7 | 11.7 | 2.7 |
| Carnitine | 164.1 | 10.8 | 127.0 | 21.7 | 9.8 |
| Methyl carnitine | 9.4 | N.D. | 3.2 | 39.0 | 12.2 |
| Trimethylglycine | 23.8 | 30.8 | 98.6 | 3447.8 | 13.9 |
| Methyl | 76.1 | 30.8 | 3.5 | 12622.1 | 20.7 |
| trimethylglycine | | | | | |
| Ethyl | 147.7 | 6.78 | 3.1 | 33.2 | 48.2 |
| trimethylglycine | | | | | |
| Propionyl | 217.4 | N.D. | 4.2 | 88.1 | N.D. |
| trimethylglycine | | | | | |

ND: not detected (below detection limit)

### INDUSTRIAL APPLICABILITY

The fermented food extract of the present invention has a significant vasodilatory effect; by comprising said extract as an active ingredient, it is possible to produce functional health foods or therapeutic pharmaceuticals for hypertension, etc.

1. A fermented food extract composition comprising at least acetylcholine, and propionylcholine and/or lactylcholine, which shows blood pressure-lowering effect and vasodilatory effect upon oral administration.

2. The extract composition according to claim 1, wherein the fermented food is one type selected from the group consisting of fermented buckwheat, black vinegar, yogurt, natto, and Japanese sake.

3. The extract composition according to claim 2, wherein the fermented food is fermented buckwheat.

4. The extract composition according to claim 1, further comprising at least one type selected from the group consisting of butyrylcholine, levulinylcholine, carnitine, methyl carnitine, trimethylglycine (betaine), methyl trimethylglycine, ethyl trimethylglycine, propionyl trimethylglycine and phosphocholine.

5. A food or pharmaceutical composition comprising, as an active ingredient, the extract composition according to claim 1.

6. The food or pharmaceutical composition according to claim 5, further comprising, as an active ingredient, at least one type selected from the group consisting of tyrosine and y-aminobutyric acid (GABA).

7. The food or pharmaceutical composition according to claim 5, further comprising at least one type selected from the group consisting of lactic acid, acetic acid, and citric acid.

8. A method of producing the fermented food extract composition according to claim 1, characterized in that the method consists of the steps comprising:
(1) suspending a lyophilizate or concentrate of fermented buckwheat in acetone, stirring the suspension by shaking, centrifuging the suspension to obtain a supernatant, and vacuum concentrating the resultant supernatant (step 1),
(2) eluting the vacuum concentrate of step 1 using a solid phase extraction cartridge filled with a carrier to which a phenyl group is bonded, with acid-containing water as an eluent (step 2),
(3) fractionating and purifying the eluate of step 2 using a column filled with a carrier to which a pentafluorophenyl group is bonded (PFP column), with acidic methanol-containing water as a mobile phase (step 3).

9. The production method according to claim 8, characterized in that:
(1) in step 2, the solid phase extraction cartridge is a GL Science InertSep® PH cartridge, and the eluent is 0.01% formic acid-containing water,
(2) in step 3, the PFP column is a YMC-Triart PFP used as a reverse phase column, and the mobile phase is water containing 0.01% formic acid-33% methanol.

10. A fermented food extract composition produced by the production method according to claim 8, characterized in that it comprises at least acetylcholine, and propionylcholine and/or lactylcholine, and shows blood pressure-lowering effect and vasodilatory effect upon oral administration.
